(19) United States
(12) Patent Application Publication
(10) Pub. No.: US 2010/0087766 A1
(43) Pub. Date: Apr. 8, 2010

(54) TOE PROTECTING DEVICE

(76) Inventor: Amelia Goodes, Los Angeles, CA (US)

Correspondence Address: Law Office of Michael N. Cohen, P.C., 9025 Wilshire Blvd., Suite 301, Beverly Hills, CA, 90211, US

(21) Appl. No.: 12/246,830

(22) Filed: Oct. 7, 2008

(51) Int. Cl. A61F 5/00

(52) U.S. Cl. 602/30

(57) ABSTRACT

A toe protecting device for supporting and keeping the toe from curling is disclosed. The device includes a thin plastic strip for placing the toe straight, a plurality of washer rings through which a toe can be inserted, a cloth that surrounds the device provides cushion and an elastic band secures the toe and the strip. A plurality of washer rings attached to the flat surface of the plastic strip is lifted so as to insert at least one of a big toe and little toe. An elastic band that surrounds the toe and the device provides sufficient grip. A plurality of objects on the strip that supports and straightens the little toe is made of lightweight plastic.

### CROSS-REFERENCE TO RELATED APPLICATIONS

Not Applicable.

### STATEMENT REGARDING FEDERALLY SPONSORED RESEARCH AND DEVELOPMENT

Not Applicable.

### FIELD OF THE INVENTION

This invention relates to toe protectors, and more particularly to a device for supporting and keeping the toe from curling and allowing a person to move around without pain.

### DISCUSSION OF RELATED ART

All over the world, people have been suffering from hammertoe, a defect that results in the contracture or bending of toes. In such deformity of toe, it appears like a hammer when looked at from the side. Hammertoe may cause pain in the toes or feet when an individual gets around due to constant rubbing against the floor and shoes. Various devices and inserts were developed to correct the deformity and to relieve pressure over painful corns. Existing devices like e-crest pads, strappings, heel cushions and heel liners, metatarsal pads, toe caps, toe separators and elastomers that utilize protection bars that encircle the toes and secured to a cast are less effective.

Typically, prior art devices for protecting the toes comprise a flexible sheet of plastic open in the back and the bottom of the foot and an adhesive tape wrapped around the tongue and base of the guard. Such rigid devices rub against the toes and require an adhesive tape to maintain it from sliding from the foot. A toe protector for orthopedic foot fixture having a rigid arcuate enclosure that is positioned over the toe fails to keep the toe from curling.

Toe protectors having a metatarsal protector made from rigid synthetic plastic material integrally incorporated into the boot prevents the wearer of the boot from walking freely, and limits the range of motion of the foot as the rigid plastic material cannot bend or flex with the contours of the foot. A toe-covering sock with elastic straps pulls the socks against the toes, which may apply painful pressure on sore toes. Traditional toe protectors made of steel and metals apply an extreme load on the toe that may cause problem when a safety shoe has to be removed from an injured foot after an accident. Similarly, toe protectors having metatarsal joint securing band that are wound around the root of the big toe limits the treatment to big toe alone.

Therefore, there is a need for a convenient, reliable, safe and separate protective device for big toe and little toe that keeps the toe from curling and allowing a person to move around without pain. Such a device would eliminate the problem caused by the rubbing of toes against the ground and uncomfortable shoes. Further, the device should provide a cushion when walking. The present invention accomplishes these objectives.

### SUMMARY OF THE INVENTION

The present invention is a toe protecting device and method of supporting and keeping the toe from rubbing against the ground and uncomfortable shoes. The device comprises a thin plastic strip for placing the toe. A portion of the washer rings is attached to the flat surface of the thin plastic strip. The strip is surrounded by a cloth that provides a cushion and gives extra comfort to the toe while walking. An elastic band is provided that surrounds the toe and the device, thereby keeping the toe from rubbing against the ground and uncomfortable shoes.

The washer rings are lifted and the toe is inserted through the washer rings. The position of the washer rings on the pad are arranged such that the rings keep the toe in a straightened position and the elastic band that surrounds the device provides sufficient grip between the toe and the strip. The present invention provides separate designs that include lightweight plastic objects on the strip to support and straighten big toe and little toe.

Other features and advantages of the present invention will become apparent from the following more detailed description, taken in conjunction with the accompanying drawings, which illustrate, by way of example, the principles of the invention.

### DESCRIPTION OF THE DRAWINGS

FIG. 1A is a perspective view of the invention, illustrating a big toe positioned in a toe protecting device;

FIG. 1B is a perspective side view of the invention, illustrating a big toe secured in a toe protecting device;

FIG. 2 is a perspective view of the invention, illustrating a little toe placed in a toe protecting device;

FIG. 3A is a perspective view of the invention, illustrating a cloth and a thin plastic strip on which washer rings is attached;

FIG. 3B is a perspective view from an angle of the invention, illustrating an elastic band;

FIG. 3C is a perspective view of the invention, illustrating a plastic strip on which the elastic bands separately surrounds the washer rings;

FIG. 3D is a perspective view of the invention, illustrating a cloth and a thin plastic strip on which the washer rings is attached for protecting the little toe; and

FIG. 3E is a perspective view of the invention, illustrating the final product that keeps the toe straight to prevent irritation while on a walk.

### DETAILED DESCRIPTION OF THE PREFERRED EMBODIMENT

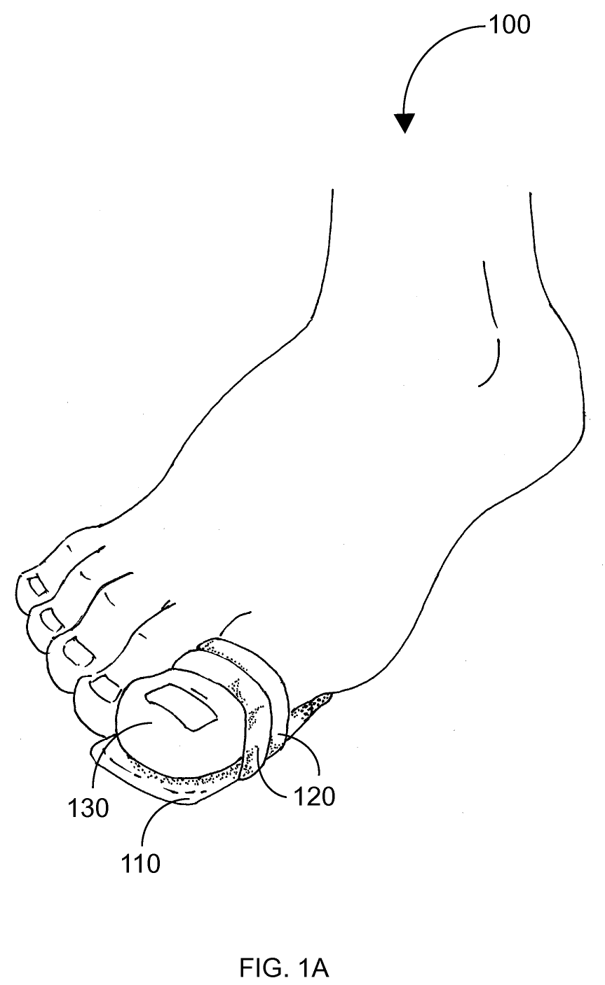

FIG. 1A shows a perspective view of a toe protecting device 100 for supporting the big toe 130 of an individual suffering from hammertoe. The device 100 keeps the big toe 130 from rubbing against the ground and uncomfortable shoes (not shown). The device 100 comprises a thin plastic strip 110 for securely placing the big toe 130. The big toe 130 is inserted through a plurality of washer rings 310 attached to the flat surface on the thin plastic strip 110. An elastic band 120 that surrounds the device 100 provides sufficient grip between the big toe 130 and the strip 110.

Preferably, referring to FIG. 1B represents a perspective view of a toe protecting device 100 for supporting the big toe 130 of an individual suffering from hammertoe is disclosed. The device 100 keeps the big toe 130 firmly in place in a straightened manner to prevent from curling or constant rubbing against the floor or shoes (not shown). The device 100 comprises a thin plastic strip 110 for placing the big toe 130. The washer rings 310 are lifted and the big toe 130 is inserted through the washer rings 310 attached to the flat surface on the thin plastic strip 110. An elastic band 120 that separately surrounds the toe 130 provides sufficient grip between the big toe 130 and the strip 110.

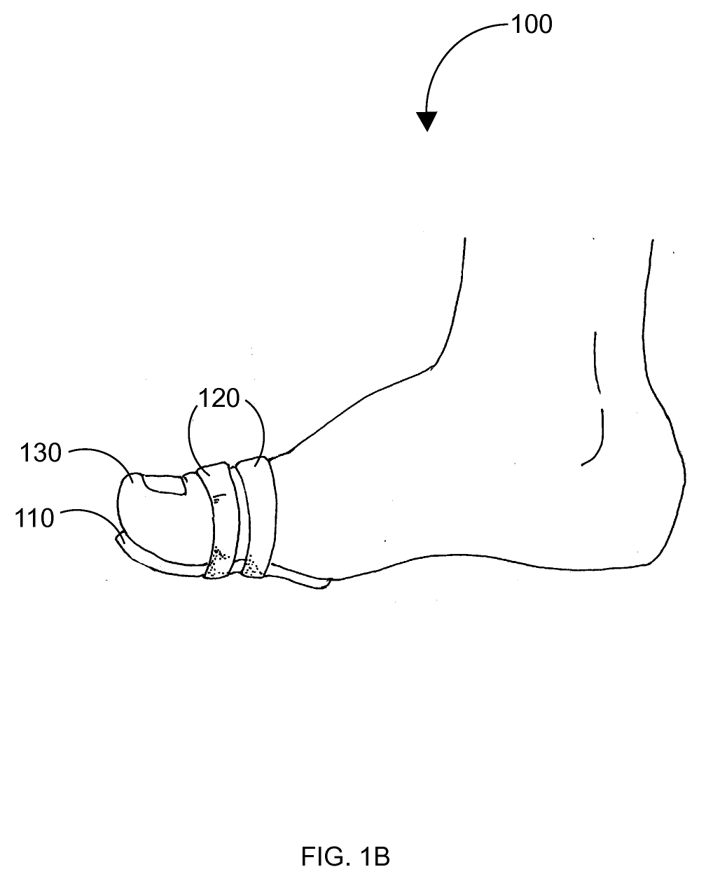

FIG. 2 shows a perspective view of a little toe protecting device 200 for supporting the little toe 210 of an individual suffering from hammertoe. The little toe 210 is placed on a thin plastic strip 610. A plurality of washer rings 620 surrounds the little toe 210. An elastic band 400 surrounds the toe 210 and the device 600.

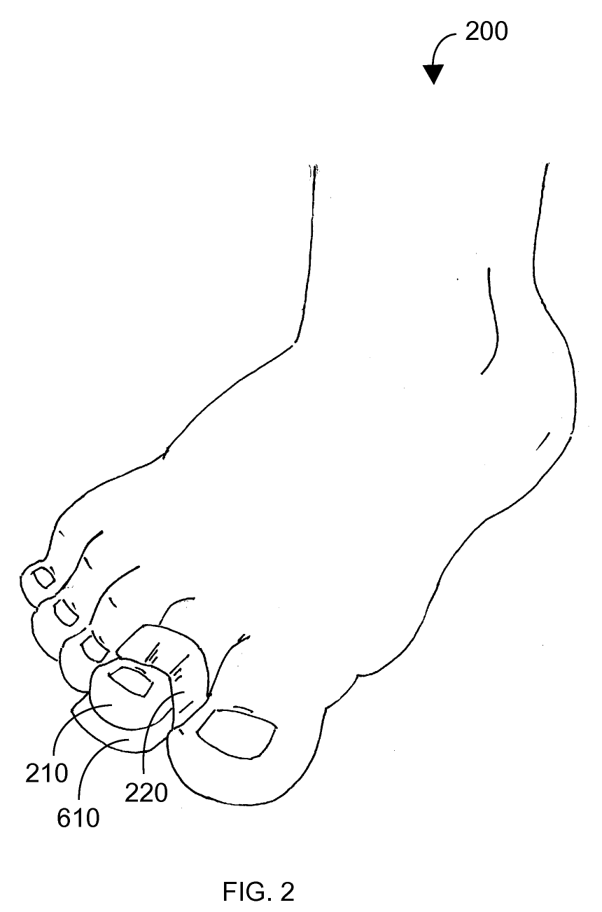

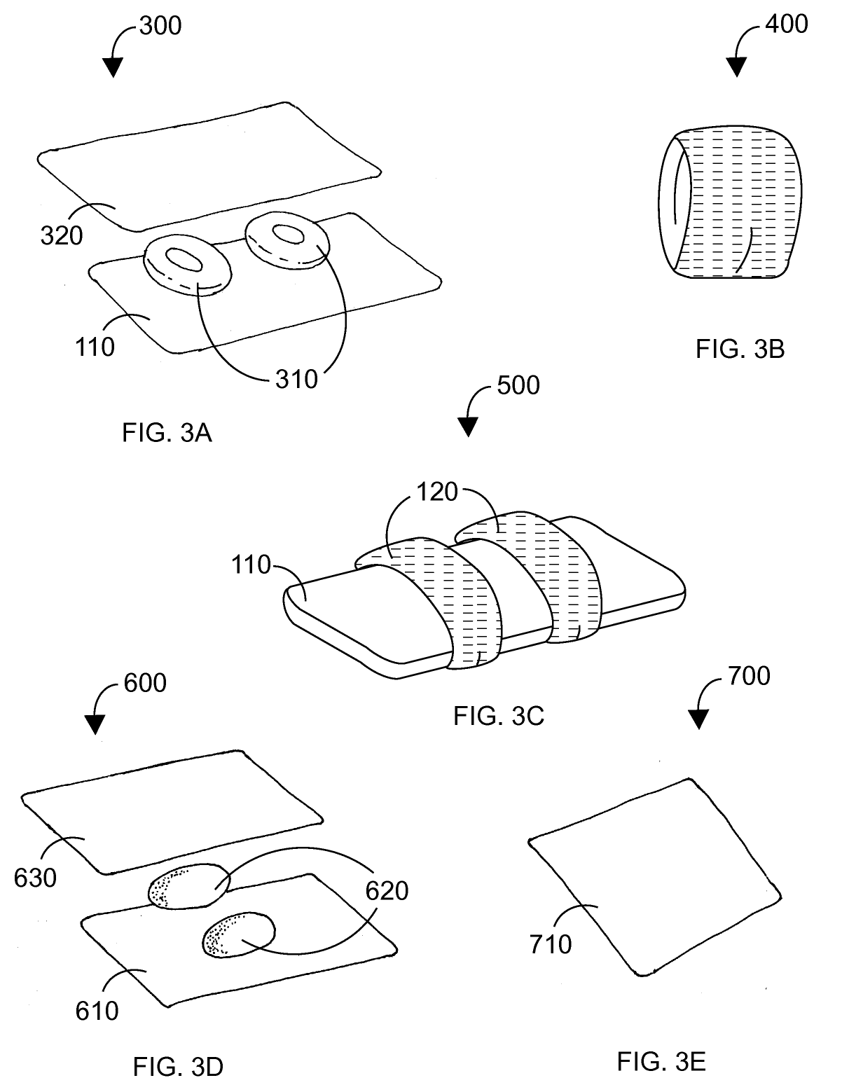

FIG. 3A shows a perspective view of a big toe protecting device 300 wherein at least one portion of the washer rings 310 is attached to the flat surface of the plastic strip 110. The washer rings 310 are lifted and the big toe 130 is inserted through the washer rings 310. A cloth 320 surrounds the washer rings 310 that are attached to the thin plastic strip 110. The cloth 320 that surrounds at least one washer ring 310 provides a cushion and gives extra comfort for the big toe 130 while walking.

FIG. 3B shows a perspective view of an elastic band 400 illustrating the band 400 that surrounds the big toe 130 and the device 300. The elastic band 400 that surrounds the big toe 130 and the device 300 keeps the toe 130 straight and provides sufficient grip.

FIG. 3C represents the perspective view of another embodiment of the toe protecting device 500 wherein a plurality of washer rings (not shown) attached to the strip 110 is surrounded separately by an elastic band 120. Separate elastic bands 120 are provided to surround each of the washer rings 310 thereby supporting and positioning the toe 130 in place. The elastic bands 120 that surrounds the washer rings 310 provides additional grip between the big toe 130 and the device 500.

FIG. 3D represents a perspective view of a little toe protecting device 600 wherein at least one portion of the washer rings 620 is attached to the flat surface of the plastic strip 610. The objects 620 on the strip 610 that support and straighten the little toe 210 is made of lightweight plastic. The washer rings 620 are lifted and the little toe 210 is inserted through the plurality of washer rings 620. A cloth 630 surrounds the plurality of washer rings 620 that is attached to the thin plastic strip 610. The cloth 630 that surrounds at least one washer ring 620 provides a cushion and helps support and straighten the little toe 210.

FIG. 3E represents a perspective view of the final product 700 that keeps the little toe straight to prevent irritation while walking. The washer rings 620 are lifted and the little toe 210 is inserted through these washer rings 620. A cloth 630 that surrounds the washers 620 provides a cushion while on a walk. At least one elastic band 400 that surrounds the little toe 210 and the device 700 provides sufficient grip.

While a particular form of the invention has been illustrated and described, it will be apparent that various modifications can be made without departing from the spirit and scope of the invention. For example, structure, material, procedure, size and the like that will fall within the scope of the device. In any event, it is understood that other materials for securing the device and the toe are also possible. Accordingly, it is not intended that the invention be limited, except as by the appended claims.

What is claimed is:

1. A toe protecting device and method of supporting and keeping the toe from rubbing against the ground and uncomfortable shoes, the toe protecting device comprising:
a thin plastic strip for placing the toe;
a plurality of washer rings attached to a flat surface on the thin plastic strip through which a toe can be inserted; and
an elastic band that surrounds the product for providing sufficient grip between the toe and the strip.

2. The toe protecting device of claim 1, wherein the thin plastic strip is placed at the base.

3. The toe protecting device of claim 1, wherein at least one portion of a plurality of washer rings is attached to the flat surface of the plastic strip.

4. The toe protecting device of claim 1, wherein a plurality of washer rings are lifted to insert the toe through a plurality of washer rings.

5. The toe protecting device of claim 1, wherein the device secures at least one of big toe and little toe.

6. The toe protecting device of claim 1, wherein each of a plurality of washer rings attached to the flat surface of the thin plastic strip is separately surrounded with at least one of a plurality of elastic bands.

7. The toe protecting device of claim 1, wherein a plurality of washer rings attached to the thin plastic strip is surrounded by a cloth.

8. The toe protecting device of claim 1, wherein the cloth that surrounds at least one washer ring provides a cushion thereby giving extra comfort for the toe while walking.

9. The toe protecting device of claim 1, wherein a plurality of objects on the strip that support and straighten the little toe is made of lightweight plastic.

10. The method of supporting the big toe in a toe protecting device to keep the toe from curling and to allow a person to move around without pain, comprising:
lifting a plurality of washer rings attached to a flat surface of a thin plastic strip;
inserting the big toe through the washer rings;
wrapping the entire assembly with a cloth; and
placing an elastic band to surround the toe and the product.

11. The method of claim 10, wherein each of a plurality of washer rings is surrounded separately by an elastic band.

12. The method of supporting the little toe in a toe protecting device to keep the toe from curling and to allow a person to move around without pain, comprising:
lifting a plurality of washer rings attached to a flat surface of a thin plastic strip;
inserting the little toe through the washer rings;
wrapping the entire assembly with a cloth; and
placing an elastic band so as to surround the toe and the product.

13. The method of claim 13, wherein a plurality of washer rings is surrounded by an elastic band.
